(19) United States
(12) Patent Application Publication
(10) Pub. No.: US 2025/0375192 A1
(43) Pub. Date: Dec. 11, 2025

(54) MEDICAL SYSTEMS, DEVICES, AND RELATED METHODS FOR DELIVERY OF FLUID AND/OR COLLECTION OF CYTOLOGY SAMPLES

(71) Applicant: BOSTON SCIENTIFIC MEDICAL DEVICE LIMITED, Galway (IE)

(72) Inventor: Deepak Kumar SHARMA, Muzaffarnagar (IN)

(73) Assignee: BOSTON SCIENTIFIC MEDICAL DEVICE LIMITED, Galway (IE)

(21) Appl. No.: 19/229,006

(22) Filed: Jun. 5, 2025

Related U.S. Application Data
(60) Provisional application No. 63/656,824, filed on Jun. 6, 2024.

(51) Int. Cl. A61B 10/02; A61M 31/00

(52) U.S. Cl. CPC A61B 10/02; A61M 31/00; A61B 2010/0208; A61B 2010/0216; A61M 2202/0468; A61M 2206/14; A61M 2210/1042

(57) ABSTRACT

Disclosed are medical devices. In an aspect, a medical device of the disclosed medical devices may comprise a tubular member defining a lumen, a threaded member extending through the lumen, and a handle including a threaded rod coupled to the threaded member, a fixed member with a threaded inner surface, and an actuator configured to translate the threaded rod distally. An interaction between the threaded rod and the threaded inner surface of the fixed member may be configured to rotate the threaded rod and the threaded member.

### CROSS-REFERENCE TO RELATED APPLICATIONS

This application claims the benefit of priority to U.S. Provisional Application No. 63/656,824, filed on Jun. 6, 2024, which is incorporated by reference herein in its entirety.

### TECHNICAL FIELD

The disclosure relates generally to systems, devices, and methods for delivering one or more fluids. More specifically, aspects of the disclosure pertain to systems, devices, and/or methods for delivering viscous fluid that may include one or more treatment agents to a target site, via medical devices, such as endoscopes.

### BACKGROUND

Bleeding ulcers or other wound sites may occur, for example, in a subject's gastrointestinal (GI) tract. For example, following another diagnostic or treatment procedure, such as endoscopic mucosal resection (EMR) or endoscopic submucosal dissection (ESD), bleeding may be desired to be prevented or treated. In another example, a line of sutures in a GI tract may be in need of reinforcement. In a further example, a fistula may require treatment. In yet another example, peroral endoscopic myotomy (POEM) sites may need closure. Therefore, a need exists for systems, devices, and methods for delivering one or more fluids.

### SUMMARY

The disclosure includes systems, devices, and methods for delivering one or more fluids such as treatment agents to a target site of a subject, for example, to help heal an ulcer and/or to perform hemostasis. The disclosed devices may also be used to obtain cytology samples. Each of the aspects disclosed herein may include one or more of the features described in connection with any of the other disclosed aspects.

In an example, a medical device may comprise: a tubular member defining a lumen; a threaded member extending through the lumen; and a handle including: a threaded rod coupled to the threaded member; a fixed member with a threaded inner surface; and an actuator configured to translate the threaded rod distally. An interaction between the threaded rod and the threaded inner surface of the fixed member may be configured to rotate the threaded rod and the threaded member.

Any of the aspects disclosed herein may include any of the following features, alone or in combination. A brush may be disposed at a distalmost end of the threaded member. The brush may be configured to rotate with the threaded member. The threaded member may have a proximal unthreaded portion. A distal end of the tubular member may have a flared shape. The actuator may be coupled to the threaded rod at a hinge. An actuation of the actuator may cause the threaded rod to translate distally. The hinge may include a bearing configured to allow for rotation of the threaded rod. The handle may include: a longitudinally extending shaft defining a handle lumen. The threaded rod and the fixed member may be disposed in the handle lumen. The handle may include: a grip extending substantially perpendicular to the longitudinally extending shaft; and an arm connecting the grip to the actuator. The handle may include a resilient member configured to bias the threaded rod toward the actuator. The resilient member may be a coil spring. The handle may include a port configured to be coupled to a fluid source. The port may be in fluid communication with the lumen of the tubular member. The threaded member may be configured to advance a fluid from the fluid source distally through the lumen of the tubular member. The threaded member may include a helical thread. Rotation of the threaded member may be configured to rotate a brush to collect a cytology sample.

In another aspect, a medical device may comprise a tubular member defining a lumen; a threaded member extending through the lumen; and a handle including: a fluid port in fluid communication with the lumen; a rod coupled to the threaded member; and an actuator configured to translate the rod distally. Translation of the rod may be configured to rotate the threaded member and to advance a fluid distally through the lumen of the tubular member.

Any of the aspects disclosed herein may include any of the following features, alone or in combination. A distal end of the threaded member may include a brush, and a distal end of the tubular member may have a flared shape.

In another aspect, a medical device may comprise a tubular member defining a lumen; a threaded member extending through the lumen; a brush disposed at a distalmost end of the threaded member; and a handle including an actuator configured to rotate the threaded member so as to move a fluid distally through the lumen to the brush.

Any of the aspects disclosed herein may include any of the following features, alone or in combination. The brush may be configured to rotate with the threaded member. The threaded member may have a proximal unthreaded portion.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a perspective view of an exemplary medical system.

FIG. 2 depicts a distal portion of a medical device of the exemplary medical system.

FIG. 3 depicts a side view of a proximal portion the exemplary medical system.

FIG. 4 depicts a perspective view of a proximal portion of the exemplary medical system.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

A fluid may be used to prevent or treat bleeding or other conditions in a GI tract. In examples, a highly viscous fluid may be delivered to the wound site(s) to form a protective layer that helps to treat or minimize delayed bleeds, potential perforations, and stricture formations. Conventional devices for fluid delivery may include one or more fluid channels, with the one or more fluid channels each having a small diameter. Highly viscous fluids may require a large amount of force or pressure to flow through narrow fluid channels. Common fluid delivery devices, e.g., syringes, generally deliver fluid when a push force, e.g., a distal force, is applied to an actuator feature of such devices. Applying a large amount of force or pressure to urge the viscous fluid distally may be difficult and/or time-consuming for the user. Additionally, applying the large amount of force or pressure may increase the risk of one or more components of the medical device deforming, breaking, or otherwise failing.

Aspects of this disclosure seek to improve and ease a user's ability to deliver a highly viscous fluid via a device delivered through a working channel of an insertion device, such as a medical scope (e.g., endoscope). Additionally, various aspects of this disclosure may help the user perform wound treatment and/or hemostasis within the subject, reduce overall procedure time, reduce overall procedure costs, etc.

Examples of fluids (e.g., biocompatible viscous fluids) that may be delivered using the devices disclosed herein include, but are not limited to, fibrin, thrombin, fluids including calcium salts, cyanoacrylates, albumin and glutaraldehyde, poly (ethylene glycol) (PEG), polyurethane, etc. Such fluids may be endoscopically delivered adhesives or other agents that help to create a protective layer that minimizes delayed bleeds, potential perforations, and stricture formations.

One of ordinary skill in the art will appreciate that the systems, devices, and methods of this disclosure may be used with a variety of biocompatible fluids and that the systems, devices, and methods of the disclosure may be applicable to various medical procedures beyond bleeding control.

Various aspects of this disclosure relate to delivery and/or mixing of various hemostatic agents at a treatment site. For example, a user may deploy multiple different hemostatic agents at the target treatment site via the systems, devices, and methods disclosed herein. In an example, a first agent (Part A) and a second agent (Part B) may be mixed together in a predefined ratio at the target treatment site. Part A and Part B may crosslink when they come in contact with one another. The cross-linked structure of the resulting combination agent (A+B) may have enhanced hemostatic properties compared to agents A or B individually. In a non-limiting example, Part A may be fibrinogen (e.g., lyophilised pooled human concentrate) and Part B may be thrombin (e.g., of bovine or human origin). Fibrinogen and thrombin may be mixed immediately before application at a target treatment site. The biocompatible fibrinogen and thrombin mixture may also contain calcium salts. One of ordinary skill in the art will appreciate that such a hemostatic mixture may be used at various locations in the human body, such as the stomach, esophagus, and colon.

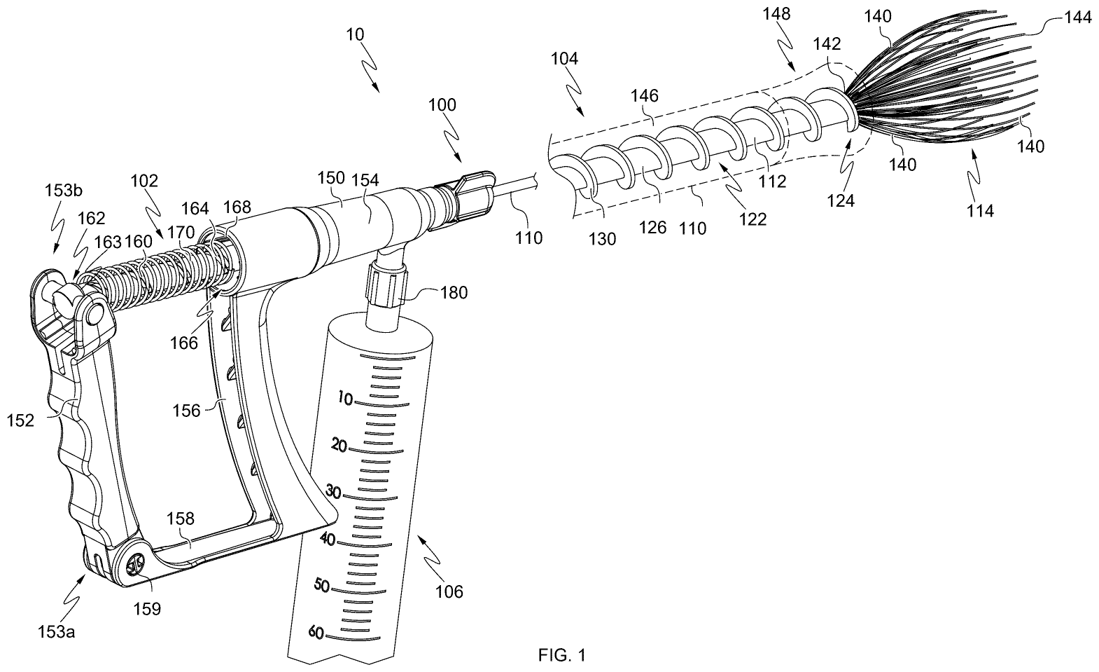

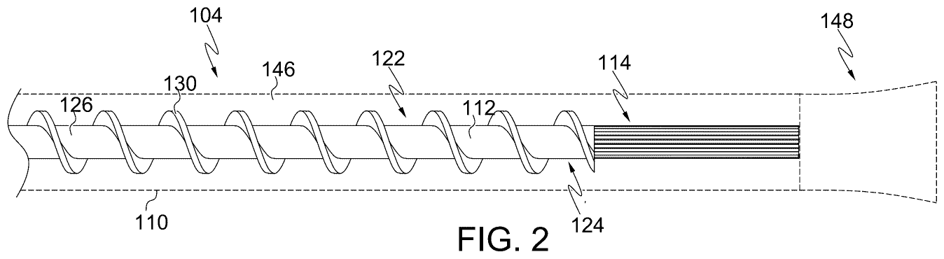

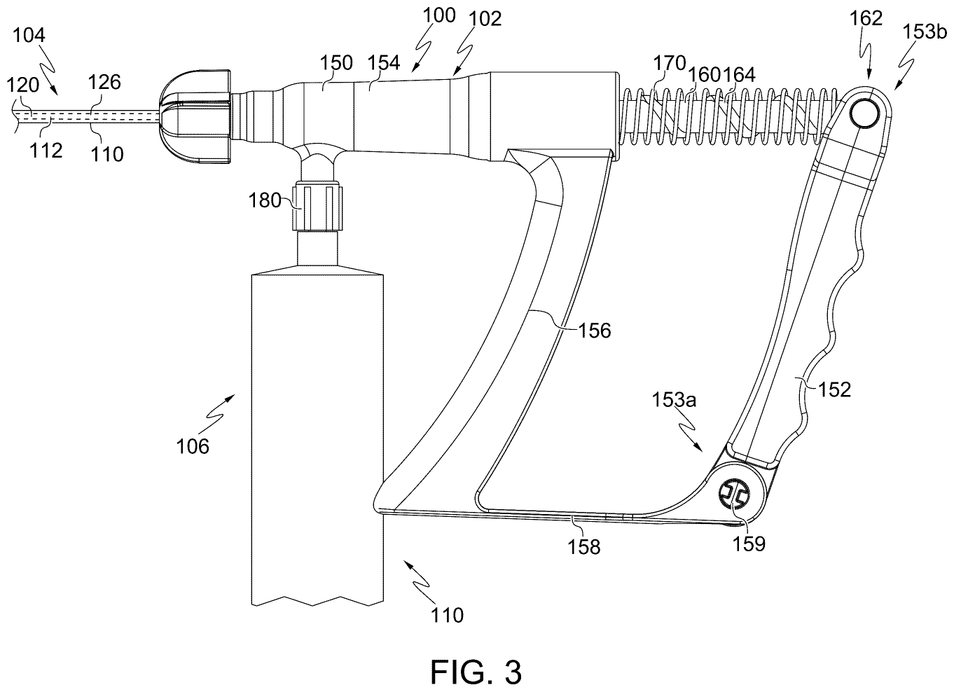

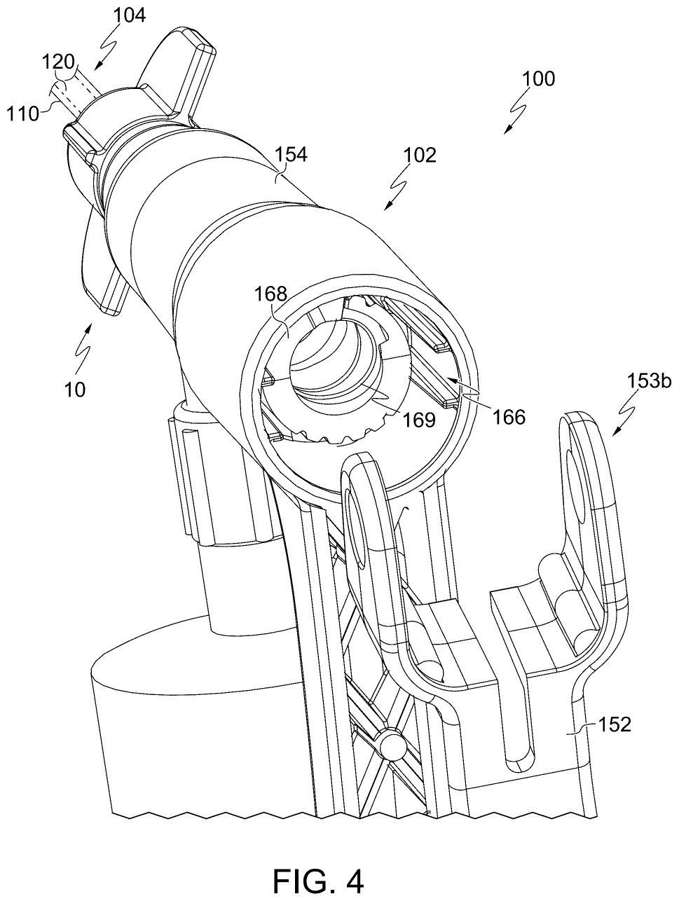

FIG. 1 depicts a perspective view of an exemplary medical system 10. Medical system 10 may include a medical device 100, and a fluid source 106. FIG. 2 depicts a distal portion of medical device 100. FIG. 3 depicts aspects of a proximal portion of medical device 100. FIG. 4 depicts a proximal portion of medical device 100 with some components omitted in order to show the details of other components. Medical device 100 may be introduced into a body lumen of a subject by an insertion device, which may include an articulable scope, such as an endoscope (not shown). It will be understood that the figures shown in the disclosure may not be to scale. For example, a distal portion of medical device 100 may be enlarged to exaggerate and emphasize certain features. As shown in FIG. 1, medical device 100 may feature a handle 102 and an insertion portion 104. Insertion portion 104 may be delivered to a treatment site (e.g., via an insertion device), and handle 102 may remain outside of a subject's body.

Insertion portion 104 may define a longitudinal axis and may extend distally from handle 102. Insertion portion 104 may move proximally and/or distally through tortuous patient anatomy via an insertion device such as an endoscope (not shown). Insertion portion 104 may include a tubular member 110 (e.g., a pipe) and a threaded member 112 within and coaxial with pipe 110. A brush 114 may be disposed at a distal end of threaded member 112.

Threaded member 112 may include a proximal portion 120 (see FIGS. 3 and 4) and a distal portion 122. Threaded member 112 may be comprised of a shaft 126 (e.g., a wire or flexible rod) and at least one helical thread 130. In some examples, there is one helical thread 130. Helical thread 130 may extend along a portion of a length of shaft 126. In some examples, helical thread 130 may extend an entire length of shaft 126. In other examples, helical thread 130 may be disposed only on distal portion 122, while proximal portion 120 may be unthreaded and not include helical thread 130. Helical thread 130 may be configured to transmit a fluid in a distal direction through pipe 110 from a proximal end of pipe 110 to a distal end of pipe 110, as discussed in further detail below.

A brush 114 may be disposed at a distalmost end 124 of threaded member 112. Brush 114 may be comprised of a plurality of filaments 140 and may be configured to receive a fluid that is transmitted through pipe 110 by helical thread 130. Filaments 140 of brush 114 may vary in quantity, cross-sectional shape, length as measured from the distalmost end 124 of threaded member 112, radius of curvature at a proximal portion, angular separation between each of the filaments 140, etc. Brush 114 may have any suitable properties (e.g., stiffness, quantity of filaments 140, etc.) for delivering a fluid and/or for collecting a cytology sample from a subject's tissue.

Some or all of brush 114, including filaments 140, may include wires, cables, or lines, etc. For example, each of filaments 140 may include a wire that has a proximal end 142 coupled to distalmost end 124 of threaded member 112 and a distal end 144 that is free (e.g., not connected to any other structures). Brush 114, including filaments 140 may be composed of a flexible material with shape-memory properties, such as nitinol or other similar material(s). In some examples, brush 114 may be replaced with a sponge or other absorbent or otherwise flexible member.

Brush 114 may include an expanded configuration (see FIG. 1). Brush 114 may be in the expanded configuration when brush 114 extends distally beyond pipe 110. In the expanded configuration, distal ends 144 of filaments 140 may flare outward and away from a centerline defined by pipe 110, such that a distance between respective distal ends 144 of filaments 140 is greater than a distance between respective proximal ends 142 of filaments 140. Distal ends 144 of filaments 140 may extend radially outward from pipe 110 while proximal ends 142 may remain radially disposed within a cylinder defined by pipe 110. Brush 114 may further include a contracted configuration (see FIG. 2). Brush 114 may be in the contracted configuration when brush 114 is retracted proximally and is disposed within pipe 110. In the contracted configuration, each of filaments 140 may extend approximately parallel to one another and approximately parallel to a central longitudinal axis of threaded member 112. In the contracted configuration, distal ends 144 and proximal ends 142 of filaments 140 may remain radially disposed within pipe 110. Brush 114 may be in the contracted configuration when the medical device 100 is being positioned within a patient's anatomy, and brush 114 may be actuated into the expanded configuration when medical device 100 arrives at a target wound site to deliver a fluid to the target wound site and/or to collect a cytology sample. The actuation of medical device 100 and the delivery of fluid to a wound site will be described in greater detail below.

Pipe 110 may be generally cylindrical and may define a lumen 146 through which threaded member 112 extends. Distalmost end 148 of pipe 110 may flare radially outwardly (e.g., to accommodate brush 114). A fluid may flow through lumen 146 upon an actuation of the medical device, the fluid flowing through lumen 146 based on a rotation of threaded member 112. The fluid may flow through lumen 146 from a fluid source 106, to be described in greater detail below.

Handle 102 may include a handle body 150 and an actuator 152. Handle body 150 may include a longitudinally extending portion 154, a grip 156, and an arm 158. Grip 156 may be shaped and configured to accommodate a hand of a user. Grip 156 may extend substantially perpendicularly from longitudinally extending portion 154. Arm 158 extends in a proximal direction from grip 156 toward actuator 152, which may be located at a position proximal than grip 156. Arm 158 may extent approximately perpendicularly to a longitudinal axis of device 100.

Actuator 152 may include ergonomic grips configured to accommodate a hand of a user. In a relaxed (unactuated) configuration, as shown in FIG. 1, actuator 152 may extend approximately parallel to grip 156, and approximately perpendicularly to arm 158. Actuator 152 and grip 156 may be configured such that a user may grasp both with a single hand, for example, with fingers on grip 156 and thumb on actuator 152, or with one hand gripping each of grip 156 and actuator 152. A first end 153a of actuator 152 may be pivotally coupled to arm 158 at a first hinge 159.

A second end 153b of actuator 152 may be pivotally coupled to a rod 160 at a second hinge 162. Actuator 152 may be configured to move (e.g., translate) rod 160 proximally and distally relative to handle body 150. Rod 160 may be integrally attached or coupled to threaded member 112. As such, threaded member 112 may be moved proximally and distally relative to pipe 110 upon actuation of actuator 152. As described in further detail below, rod 160 may further be rotated as it is actuated, such that rod 160 rotates relative to handle body 150 and threaded member 112 rotates relative to pipe 110 as rod 160 and threaded member 112 are actuated by actuator 152.

To achieve this rotation, rod 160 may include at least one thread 164 (e.g., one or more helical threads) on an outer surface of rod 160. Rod 160 may extend into a lumen 166 within longitudinally extending portion 154 of handle body 150. A nut 168 (or other similar fixed member or receptacle) may be fixedly disposed within lumen 166 of handle body 150. Nut 168 may have a threaded inner surface 169 that interacts with thread 164 on the outer surface of rod 160 (see FIG. 4, in which rod 160 is omitted to show details of nut 168) to receive rod 160 within lumen 166 and to cause rod 160 to rotate as it is translated axially in a distal direction by a force applied to actuator 152.

A resilient member 170 may be disposed between handle body 150 and actuator 152. Resilient member 170 may bias actuator 152 into an open, unactuated position, as shown in FIG. 1, the unactuated position being when actuator 152 is most distant from grip 156. In some examples, resilient member 170 may be a coil spring extending from handle body 150 to second end 153b of actuator 152. Resilient member 170 may be disposed coaxially surrounding rod 160 and may apply its biasing force in a proximal direction as shown in FIG. 1.

In the unactuated position, actuator 152 has been rotated about first hinge 159 such that second end 153B of actuator 152 is at a most proximal position. To transition actuator 152 to the actuated position, a force may be applied to compress resilient member 170 and move second end 153B in a distal direction toward handle body 150. Handle 102 may include additional or alternative actuators. One of ordinary skill in the art will appreciate that various actuators may be implemented at handle 102. It will be appreciated that the systems, devices, and methods of the disclosure may be adapted to other actuators than those shown.

A port 180 may be disposed on handle body 150 that is configured to couple handle 102 to a fluid source 106. Fluid source 106 may be configured to dispense fluid into lumen 146 via port 180. Port 180 may be in fluid communication with lumen 146. In some examples, fluid source 106 may be removably connected to port 180. A first fluid source 106 may be connected to port 180, dispense a first fluid (e.g., a first agent, or part A) into lumen 146, and then be disconnected from port 180. Subsequently, a second fluid source 106 may be connected to port 180, and dispense a second fluid (e.g, a second agent, or part B) into lumen 146. In other examples, fluid source 106 may dispense a combination agent (A+B) into lumen 146. Fluid source 106 may be a syringe or any other suitable source of fluid (e.g., bag, bottle, etc.)

Operation of medical device 100 and further features of medical device 100 are described below. Fluid source 106 may be connected to port 180 on handle body 150. A fluid supplied via fluid source 106 may be a fluid used to prevent or treat bleeding or other conditions in a GI tract. In examples, the fluid may be a highly viscous fluid and may be delivered to wound site(s) via insertion portion 104 to form a protective layer that helps to treat or minimize delayed bleeds, potential perforations, and stricture formations.

Pipe 110 may be directed to a wound site, for example, via the working channel of an insertion device, such as a scope (not shown). After fluid source 106 is connected to port 180, fluid source 106 is in fluid communication with lumen 146 of pipe 110 via port 180. Lumen 146 includes therein threaded member 112, which, as discussed above, may be comprised of a shaft 126 (e.g., a wire) and at least one helical thread 130. Threaded member 112 may be actuated to rotate by actuation of actuator 152, and upon a rotation of threaded member 112, at least one helical thread 130 may advance a fluid supplied to lumen 146 from fluid source 106 in a distal direction, advancing the fluid from fluid source 106 to brush 114 disposed at distalmost end 124 of threaded member 112. Threaded member 112 is rotated via actuator 152, as described below.

In some aspects, a plunger of fluid source may be used to deliver an amount of fluid into lumen 146. However, the force on the plunger need not be sufficient to cause the fluid to travel to distalmost end 148 of tubular member 110. In alternatives, actuation of actuator 152 may cause a fluid flow from fluid source 106 into lumen 146 (e.g., by generating negative pressure).

A user may actuate actuator 152 by pushing actuator 152 toward grip 156, which causes actuator 152 to rotate about hinge 159, compressing resilient member 170 and pushing rod 160 distally via rotation of actuator 152 about hinge 162. As discussed above, grip 156 and actuator 152 may be ergonomically designed to accommodate a user's hand. For example, a user's fingers may wrap around grip 156 while a user uses their thumb to push actuator 152 toward grip 156 by squeezing their hand.

As rod 160 is moved distally, threaded engagement between threads 164 on rod 160 and threads on threaded inner surface 169 of stationary nut 168 causes rod 160 to rotate. Hinge 162 may include a bearing 163 to allow rotation of rod 160 as it is translated. Rod 160 may be integrally connected or coupled to threaded member 112, such that rotation and translation of rod 160 results in rotation and translation of threaded member 112.

The fluid that enters lumen 146 from fluid source 106 may be transferred in a distal direction along lumen 146 by rotation of helical thread 130, which transmits fluid in the distal direction in a manner similar to an Archimedes screw. When fluid reaches distalmost end 124 of threaded member 112/distalmost end 148 of tubular member 110, the fluid is deposited onto/delivered into brush 114. Brush 114 may be moved distally with threaded member 112 upon actuation of actuator 152. As such, brush 114 may be in a non-expanded, or contracted, state within lumen 146 in a starting configuration, in which filaments 140 of brush 114 are generally parallel to each other, as shown in FIG. 4, and brush 114 may be pushed out of lumen 146 upon an actuation of actuator 152. When brush 114 emerges from lumen 146 of insertion portion 104, filaments 140 of brush 114 may expand into the expanded configuration shown in and described with reference to FIG. 1. In alternatives, device 100 may include one or more features to inhibit distal movement of brush 114, while threaded member 112 rotates during an entirety or a portion of an actuation range of actuator 152.

Highly viscous fluids may conventionally require a large amount of force or pressure to flow through narrow fluid channels. Common fluid delivery devices, e.g., syringes, generally deliver fluid when a distal force is applied to an actuator feature of such devices. In contrast, helical thread 130 may transmit fluid to brush 114 with less force required on actuator 152 than on actuators of conventional devices.

Brush 114 may rotate with threaded member 112 during an actuation of actuator 152, such that filaments 140 may spray fluid onto a target wound site via the rotating action of filaments 140 of brush 114. Additionally or alternatively, brush 114 may be used to paint fluid onto a target site. Actuator 152 may be actuated multiple times to continue rotation of threaded member 112 and brush 114 to continue providing fluid to brush 114 and continue application of a fluid onto a wound site via the rotation of brush 114.

In some examples, the fluid in fluid source 106 is a mixture of a first agent and a second agent that is mixed together in a predefined ratio. In other examples, only the first agent is applied via a first fluid source 106. Handle 102 may be disposed outside of a patient's body while in use, such that port 180 is accessible during the medical procedure. First fluid source 106 may be removed and replaced with a second fluid source 106 without removal of the medical device 100 from a patient's body. First fluid source may be a first agent (Part A) and second fluid source may be a second agent (Part B). As discussed above, first agent (Part A) and second agent (Part B) may be mixed together in a predefined ratio at the target treatment site. Part A and Part B may crosslink when they come in contact with one another. The cross-linked structure of the resulting combination agent (A+B) may have enhanced hemostatic properties compared to agents A or B individually. In alternatives, handle 102 may include a plurality of ports 180 to allow for delivery of a plurality of fluids, which may interact as described above.

In some examples, filaments 140 of brush 114 may also be used to grasp and remove objects and/or to collect a tissue sample. In some examples, the rotation of filaments 140 may be used for obtaining cytology samples from a target site, such as a lesion. Alternatively, user may position the brush 114 to encapsulate or envelope a target tissue or object for recovery. When the target (e.g., object, target tissue, or cytology sample) is procured, an actuation of actuator 152 may retract the brush 114 into the pipe 110, contracting the brush 114 around the target tissue or object, and pulling the target tissue or object into pipe 110.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device comprising:
a tubular member defining a lumen;
a threaded member extending through the lumen; and
a handle including:
a threaded rod coupled to the threaded member;
a fixed member with a threaded inner surface; and
an actuator configured to translate the threaded rod distally;
wherein an interaction between the threaded rod and the threaded inner surface of the fixed member is configured to rotate the threaded rod and the threaded member.

2. The medical device of claim 1, comprising:
a brush disposed at a distalmost end of the threaded member.

3. The medical device of claim 2, wherein the brush is configured to rotate with the threaded member.

4. The medical device of claim 1, wherein the threaded member has a proximal unthreaded portion.

5. The medical device of claim 1, wherein a distal end of the tubular member has a flared shape.

6. The medical device of claim 1, wherein the actuator is coupled to the threaded rod at a hinge, and an actuation of the actuator causes the threaded rod to translate distally.

7. The medical device of claim 6, wherein the hinge includes a bearing configured to allow for rotation of the threaded rod.

8. The medical device of claim 1, wherein the handle includes:
a longitudinally extending shaft defining a handle lumen, wherein the threaded rod and the fixed member are disposed in the handle lumen.

9. The medical device of claim 8, wherein the handle includes:
a grip extending substantially perpendicular to the longitudinally extending shaft; and
an arm connecting the grip to the actuator.

10. The medical device of claim 1, wherein the handle includes:
a resilient member configured to bias the threaded rod toward the actuator.

11. The medical device of claim 10, wherein the resilient member is a coil spring.

12. The medical device of claim 1, wherein the handle includes:
a port configured to be coupled to a fluid source, the port in fluid communication with the lumen of the tubular member.

13. The medical device of claim 12, wherein the threaded member is configured to advance a fluid from the fluid source distally through the lumen of the tubular member.

14. The medical device of claim 1, wherein the threaded member includes a helical thread.

15. The medical device of claim 1, wherein rotation of the threaded member is configured to rotate a brush to collect a cytology sample.

16. A medical device comprising:
a tubular member defining a lumen;
a threaded member extending through the lumen; and
a handle including:
a fluid port in fluid communication with the lumen;
a rod coupled to the threaded member; and
an actuator configured to translate the rod distally;
wherein translation of the rod is configured to rotate the threaded member and to advance a fluid distally through the lumen of the tubular member.

17. The medical system of claim 16, wherein a distal end of the threaded member includes a brush, and a distal end of the tubular member has a flared shape.

18. A medical device comprising:
a tubular member defining a lumen;
a threaded member extending through the lumen;
a brush disposed at a distalmost end of the threaded member; and
a handle including an actuator configured to rotate the threaded member so as to move a fluid distally through the lumen to the brush.

19. The medical device of claim 18, wherein the brush is configured to rotate with the threaded member.

20. The medical device of claim 18, wherein the threaded member has a proximal unthreaded portion.
